(19) United States
(12) Patent Application Publication
(10) Pub. No.: US 2013/0184359 A1
(43) Pub. Date: Jul. 18, 2013

(54) Processes for Extracting Colors from Hibiscus Plants

(71) Applicant: Karim Nafisi-Movaghar, Walnut Creek, CA (US)

(72) Inventor: Karim Nafisi-Movaghar, Walnut Creek, CA (US)

(21) Appl. No.: 13/349,646

(22) Filed: Jan. 13, 2012

(51) Int. Cl. A61K 47/46; A61K 8/97; A23L 1/28; A23L 1/27

(52) U.S. Cl. 514/783; 426/52; 435/267; 426/431

(57) ABSTRACT

Processes are provided for extracting color comprising anthocyanin and antioxidants from hibiscus flowers. Dried or wet flowers or flower particles are soaked in water to release color from the flowers. The released anthocyanin and antioxidants are suspended in water and separated from the solid components using centrifugation, filtration, water flotation, pressing, or any combination of separation techniques. The hibiscus color may be concentrated into a color concentrate and stabilized with a stabilizing agent, such as, for example, a sugar alcohol having a 2 to 12 carbon backbone, at a pH value in the range of about 2 to about 6. Residual hibiscus color in the solid hibiscus components may be collected by mixing the components with water and then separating the residual hibiscus color using any one or more separation techniques. The residual hibiscus color may also be concentrated and stabilized with the hibiscus color or the color concentrate.

### FIELD OF THE INVENTION

The invention relates to apparatus, systems, and processes for extracting stable natural color concentrates from hibiscus plants.

### BACKGROUND OF THE INVENTION

Throughout history, dyes and coloring agents have been used for a variety of purposes. For example, green copper salts extracted from miners were used as an eye shadow by the Egyptians as early as 5000 B.C. Similarly, henna was and is still used as a hair and fingernail colorant in the Middle East, Asia, and other parts of the world. A variety of plant and animal dyes have been used to dye textiles, and certain colors were valued more highly than others. R. A. Donkin, The Insect Dyes of Western and West-Central Asia, Anthropes, Herausgeber Anthtopos-Institut, 5205 St. Augustin, Deutschland. Vol. 72 (1977). Presently, colors are used extensively in textiles, foods, drinks, drugs, and cosmetics.

Coloring foodstuffs and condiments dates back at least 500 years. In general, colors are added to food products to improve appearance. Some food producers believe that the color of a food product is more influential on consumers than the flavor of the food product. To consumers, an off-color food suggests an inferior product. Color may be added to a food product to make the product recognizable or to compensate for color loss during food processing.

Until the middle of the nineteenth century, most of the colorants used in food, drugs, and cosmetics were obtained from natural sources.

One problem with many natural pigments found in plants or animals is their relative instability. Changes or deviations from natural conditions of these pigments can cause physical and chemical changes to the colors they exhibit. This includes pH changes or the effect of light or temperature, particularly present during the processing necessary to preserve products for an extended period of time, such as two to three years.

The instability of natural pigments led to the development of synthetic colors. The first synthetic dye was developed by a British chemist in 1856. Use of the first synthetic dye in food occurred with dairy products in the United States, and synthetic colors were allowed in 1886 for butter and in 1896 for cheese. By the year 1900, synthetic colorants were used in a wide range of foods, such as ice cream, candy, ketchup, jellies, noodles, wine, and many more.

In the 1950s, the effect of synthetic colorants on human health was first recognized when animal studies implicated some colorants as causing health concerns. As a result, some colorants are no longer permitted in food.

Of particular interest to the invention is the status of red colorings allowed in foods. Within the last thirty years at least four different FD&C Red colors have been delisted by the U.S. Government and are no longer permitted in food. A recent example of a delisted color is FD&C Red. No. 3 which is a Xanthene dye with a range of application from lipstick to candy to dyed cherries. FD&C Red. No. 3 has a maximum absorbency wavelength at about 520 nm and is chemically very stable. It precipitates under acidic conditions. However, in January of 1990, the U.S. Food and Drug Administration (FDA) announced the formal banning of FD&C Red No. 3 in drugs and cosmetics based on the required compliance with the Delany Clause of the Food, Drug and Cosmetic Act. This ban, however, does not yet disapprove “permanently” listed uses of the food dye from ingested drugs and foods.

Studies show that certain synthetic dyes pose adverse health effects. For example, some synthetic dyes, such as FD&C Red No. 3, are recognized as carcinogenic. In recent years, public interest groups and private organizations have raised concerns that certain synthetic dyes may cause hyperactivity and other behavior disorders in children. The risk posed by synthetic and some natural dyes in commercial use with food continues to be investigated.

Processed and unprocessed natural and organic foods are in high demand by health conscious consumers. Processed foods with natural and organic ingredients appeal to consumers more than processed foods containing many synthetic ingredients. Consequently, broad commercial use of synthetic colors in foods has caused consumers to focus on food ingredients when selecting food to consume. The possible adverse health effects of synthetic dyes in children has caused parents to avoid purchasing foods with synthetic ingredients for their children to eat. Consumer awareness of food ingredients drives sales of natural and organic foods. The use of natural food colorings in food appeals to consumers. Food manufacturers are evolving food processing techniques to substitute natural food colorings for synthetic dyes to meet consumer demand. For example, replacing red dye No. 3 in some foods with an insect origin dye (carmine) is described in Meloan et al. Histochemie 37, 87 (1971). Another example is producing natural yellow and orange color from tomato skin for use as a replacement of FD&C Yellow No. 5 and Yellow No. 6. Karim Nafisi, U.S. Pat. No. 4,781,936.

One natural source of food coloring is anthocyanin, which is naturally occurring in the flower of hibiscus plants. The representative polyphenolic colorant anthocyanin is a subgroup of flavonoids that are water-soluble glycosides of anthocyanidins. Anthocyanins contain a C₆C₃C₆ backbone and cover a broad range of the color spectrum including blue, purple, violet, magenta, red, and orange. Although other flavonoids release colors, anthocyanins are the most broadly distributed pigment in plants. Anthocyanins differ in the number of hydroxyl and/or methoxy groups present, and sugars, such as, glucose, galactose, arabinose, and xylose, are attached to the 3 position in the C ring. When the attached sugars are hydrolyzed into aglycone and sugar, the aglycone is referred to as an anthocyanidin, which is another color source along with anthocyanin. The color of anthocyanins and anthocyanidins results from excitation of a molecule by light, and the strength of the color is determined by the relative electron mobility in the structures. Since the two colorings have many conjugated double bonds which are readily excitable, the compounds readily release color when exposed to light. Anthocyanins and anthocyanidins serve as natural food additives or ingredients and also contribute health benefits as a rich source of antioxidants.

Certain processes exist for extracting food grade coloring from flowers, including hibiscus flowers. For example, U.S. Pat. No. 5,704,950 teaches extracting food colors from hibiscus using heated water. The solution obtained by heat treatment contains color from the flower. The pH value of the solution is adjusted to about 7.5-8 using alkaline amino acid. Then, the solution is boiled for 1-2 hours to mature the color extract. After being adjusted to mildly acidic, the extract must be filtered, mixed with a polysaccharide solution, and spray dried into a powder for use as a food coloring.

Cold processing may be used to extract color from hibiscus flowers. For example, U.S. Pat. No. 6,730,243 teaches lyophilizing hibiscus flower petals to create anthocyanin-containing powders. Another cold extraction process taught by Olukemi et al., Elec. J. Envtl. Agric. & Food Che., 4(1), 858-862 (2005) includes collecting anthocyanins from hibiscus flowers by mixing flower material with methanol and 0.1% glacial acetic acid at 19° C. to create a cold anthocyanin-containing solution.

Hot processing may also be used to extract color from hibiscus plant material. U.S. Pat. No. 6,730,243 teaches hot air drying hibiscus plant material and then purifying the dried material into an anthocyanin powder. Another hot extraction process taught by Olukemi includes mixing plant material with methanol and 0.1% glacial acetic acid and then incubating the mixture at 60° C. for 30 minutes. The extract is filtered and concentrated under reduced pressure. Yet another example of hot processing taught by Chumsri et al. Songklanakarin J. Sci. Technol. 30 (Suppl. 1), 133-139 (2008) uses water extraction of anthocyanin from fresh hibiscus flowers using a juice extractor or filter to separate color from hibiscus flower in water at about 60° C. and concentrating the extract under vacuum or at atmospheric pressure.

The disclosed processes present drawbacks for extracting anthocyanin from hibiscus plant material. Pollution-generating solvents must be used in hot and cold extraction processes. Some of the processes use non-food-grade solvents, such as methanol. Other processes require time and temperature sensitive processing techniques. Although lyophilizing or hot air drying hibiscus flowers is simple, further processing is required to extract anthocyanin from the dried plant product and to purify it for use.

The description of the prior art provided with this disclosure highlights the need for improved processes for extracting color from hibiscus flowers in such a way that (1) the hibiscus color is extracted with a water-based process without pollution-generating solvents or chemicals; (2) the hibiscus color is stable and remains stable in adverse conditions, such as exposure to light, low pH, and high temperature; (3) the process does not pose risk to the environment; (4) the hibiscus color may be extracted from wet or dry hibiscus flower; (5) the hibiscus color is acid resistant and suitable for coloring acidified fruits and vegetables without deterioration of the color; and (6) the hibiscus color is useful for coloring food, drink, cosmetics, drugs, and the like.

### BRIEF DESCRIPTION OF THE DRAWINGS

Additional aspects, features, and advantages of the invention, as to its operation and use, will be understood and become more readily apparent when the invention is considered in light of the following description of illustrative embodiments made in conjunction with the accompanying drawings, wherein:

FIG. 1 is a schematic diagram of a process for extracting color from hibiscus flowers.

FIG. 2 is a schematic diagram of a batch process for extracting color from hibiscus flowers.

FIG. 3 is a schematic diagram of a continuous solvent extraction process for extracting color from hibiscus flowers.

### DETAILED DESCRIPTION OF EMBODIMENTS OF THE INVENTION

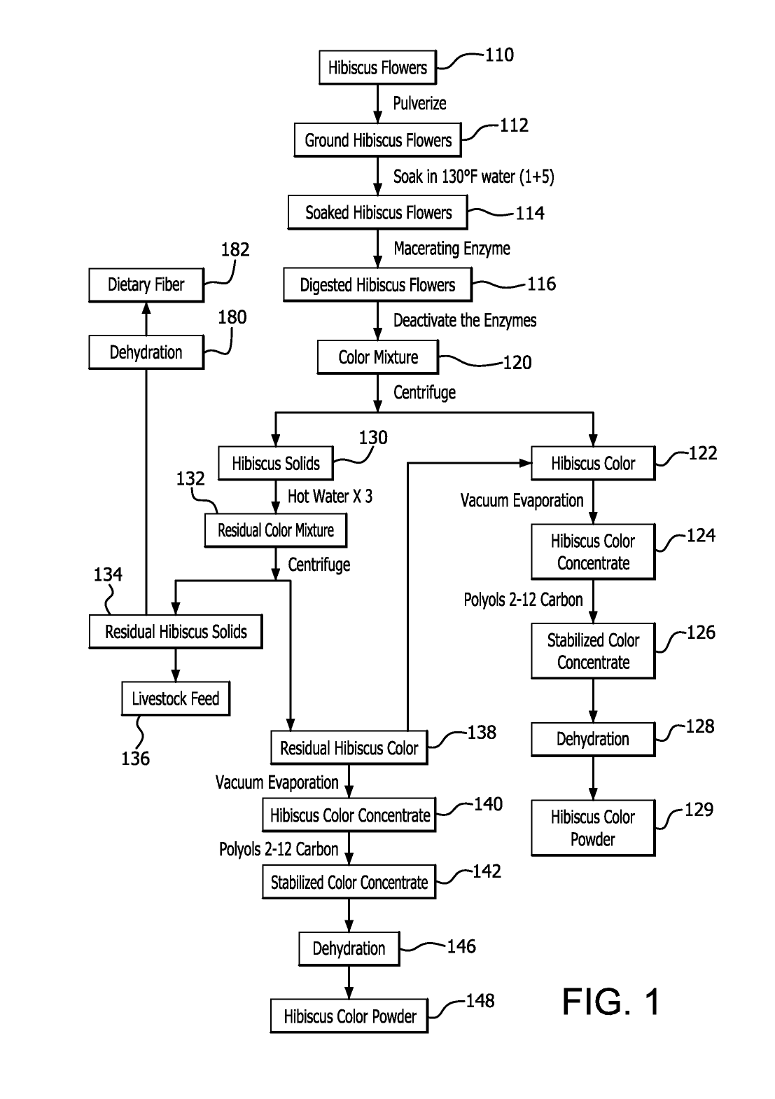

Illustrative and alternative embodiments of processes to extract a stable natural color from hibiscus flowers will be discussed with reference to FIGS. 1-3.

For this disclosure, the term “hibiscus” takes on its ordinary meaning to refer generally to a genus of flowering plants in the mallow family, Malvaceae. An illustrative and non-limiting example of hibiscus is Hibiscus rosa-sinensis. The term “hibiscus flower” or “flower” takes on its general meaning to refer to the flower of hibiscus, also called a calyx, sepal, or roselle.

The terms “hibiscus color,” “color,” and “coloring” mean (1) a solution of compounds comprising one or more of (a) organic acids (such as, for example, citric acid, malic acid, oxalic acid) and/or inorganic acids, and (b) antioxidants, including, but not limited to, anthocyanin and carotenoids dissolved in a solvent, such as, for example, water or other suitable solvent including a stabilizing agent, and (2) in the event that substantially all solvent is removed from the solution of compounds, a powder or paste of the dried compounds, which may include bulking agents as an additive.

The term “acid” is used to refer generally to organic acids and inorganic acids. The terms “food” and “drink” are used individually in reference to their ordinary plain and ordinary meanings

The invention provides processes to extract natural hibiscus color from hibiscus flowers. A purpose of the invention is to produce colors that are stable and remain stable in adverse conditions, such as exposure to light, low pH, and high temperature. Another purpose of the invention is to use water, not chemicals, with or without enzymes added to the water, for color extraction and purification to achieve a substantial yield of coloring. A further purpose of the invention is to produce an acid resistant product suitable for coloring acidified fruits and vegetables without deterioration of the color. The hibiscus color may also be used as an ingredient or colorant for food, drink, cosmetics, drugs, and the like. The hibiscus color provides health benefits which derive from its naturally-occurring antioxidant characteristics.

The processes of the invention may take place in a large scale commercial operation whereby each step may be automated with commercial machinery, carried out manually, or executed with some combination of both.

In an illustrative embodiment of the invention shown in FIG. 1, hibiscus flowers are harvested from hibiscus plants and prepared for processing to extract hibiscus color from the harvested flowers as shown in FIGS. 1-3. Hibiscus flowers 110 are dried with industrial drying equipment. In embodiments, hibiscus flowers are dried 110 with or without heat at atmospheric pressure or at a reduced pressure, such as, for example, vacuum applied. Industrial drying methods such as, for example, freeze drying, drum drying, Infra Red radiation, or by other industrial drying processes, are used to dry hibiscus flowers 110. Fresh hibiscus flowers, also referred to as “wet” flowers, may be processed without being dried, as an alternative to processing dried flowers. Fresh or dried hibiscus flowers may comprise between about 3% to about 90% water by weight.

Referring to FIG. 1, wet or dried hibiscus flowers 110 are pulverized or ground with industrial equipment into ground hibiscus flowers 112. Industrial equipment, such as, for example, a Fitzmill or the like may be used to pulverize or grind hibiscus flowers. Hibiscus flowers 110 may be ground into particles ranging from about 0.1 mm to about 2.0 mm in size. In an alternative embodiment, wet or dried hibiscus flowers 110 may also be left intact at this phase of processing rather than being ground or pulverized. Intact or ground 112—wet or dry—hibiscus flowers may be maintained in a vessel or the like at a temperature ranging from about 35° F. to about 210° F. for a time period of about 5 minutes up to about 1 year. In various embodiments, pressure exerted on the vessel may range from about atmospheric pressure up to about 20 psi. Alternatively, the vessel may be under vacuum.

Intact or ground hibiscus flowers 112 are soaked in water for a desired time and temperature to prepare the flowers to extract hibiscus color. Hibiscus flowers are soaked at a weight ratio of flower to water from about 1:1 to about 1:50 and, in a specific embodiment, about 1:5. The soak time may range from about one minute to about 20 minutes or longer. During the soak, the temperature of the mixture of water and hibiscus flowers ranges from about 35° F. to about 210° F. In a specific embodiment, the soak temperature is about 160° F. The time and temperature variables of the soak have an indirect relationship. For example, soak time may decrease at higher temperatures and may increase at lower temperatures. The soaking creates a mixture of soaked—intact or ground—hibiscus flowers 114 as depicted in the illustrative process of the invention in FIG. 1.

Referring to FIG. 1, soaked—intact or ground—hibiscus flowers 114 may be digested with macerating enzyme(s) into a substantially liquefied state. Macerating enzymes may comprise any one or combination of pectinase, cellulase, hemicellulase, or the like at desired concentrations. Macerating enzymes are introduced to soaked—intact or ground—hibiscus flowers 114 at a rate of about 0.05% to about 1% by weight at a temperature in a range of about 40° F. to about 150° F. In an embodiment, the temperature is about 130° F. The mixture of macerating enzymes with soaked hibiscus flowers 114 is held for a time ranging from about one minute to about 20 hours to allow the enzymes to partially or wholly liquefy soaked hibiscus flowers 114, 116. The mixture of macerating enzymes with the hibiscus flowers is then heated to a temperature at about 165° F. or higher to deactivate enzyme activity. The liquefied hibiscus flowers—now referred to as color mixture 120—is cooled to about 35° F. to about 150° F. and is stored or immediately transferred to the next phase of processing. In an alternative embodiment, soaked hibiscus flowers 114, which are either intact or ground, are not digested with macerating enzymes and may be stored or immediately transferred to the next phases of processing. Referring again to FIG. 1, hibiscus color 122 and hibiscus solids 130 may then be processed separately from one another into desired end products, for example, stabilized color concentrates 126, 142, hibiscus color powders 129, 148, livestock feed 136, dietary fiber 182, or waste discharge.

Referring now to FIG. 1, hibiscus solids 130 may be combined with water and heated to create a residual color mixture 132. Residual color mixture 132 may comprise a weight ratio of hibiscus solids 130 to water from about 1:1 to about 1:50, and in a specific embodiment, about 1:5. The mixture is heated to a temperature in a range of about 35° F. to about 210° F. between about 1 minute to about 60 minutes. In an embodiment, hibiscus solids 130, which are combined with water at a desired weight ratio, are heated to a temperature of about 155° F. for about 20 minutes. The heating step may be repeated up to three times or more to maximize extraction of residual hibiscus color present in hibiscus solids 130. In an alternative embodiment, a solvent including, but not limited to, any solvent described as useful for color extraction in connection with countercurrent extraction processes of the invention, may be added to the water before, during, or after heating to create residual color mixture 132. The concentration of solvent may range from about 1% up to about 95%. In an embodiment, this concentration may range from about 50% up to about 95%.

Residual color mixture 132 may be processed to separate residual hibiscus color 138 from residual hibiscus solids 134 as shown in FIG. 1. Centrifugation may be used to separate the solution of residual hibiscus color 138 from residual hibiscus solids 134. In alternative embodiments, pressing, water flotation, or filtration may be used to separate the solution of residual hibiscus color 138 from residual hibiscus solids 134. The solution of residual hibiscus color 138 may be combined with hibiscus color 122 for processing to concentrate and stabilize color into hibiscus color concentrate 124. In an alternative embodiment, the solution of residual hibiscus color 138 may be processed to concentrate it into hibiscus color concentrate 140 and stabilize it with an agent (i.e., Polyol or the like) into stabilize color concentrate 142, and optionally be further processed with dehydration 146 into a substantially desiccated hibiscus color powder 148.

Residual hibiscus solids 134 may be discarded as waste, processed into livestock feed 136, or dehydrated 180 into dietary fiber 182 as shown in FIG. 1. Solids 134 may be dehydrated by industrial drying methods into desiccated hibiscus solids. The drying method may include spray drying among other drying methods. The desiccated hibiscus solids which may comprise intact flowers, pulverized flowers, or enzymatically-digested flower parts, provide a source of dietary fiber 182. Dehydrated hibiscus solids may be cut or ground into any desirable particle size, including a powder, and used as an ingredient in food, drink including juices and herbal teas, sold as a stand alone product, or used in other applications where dietary fiber is a desired ingredient.

Referring to FIG. 1, the solution of hibiscus color 122—with or without residual hibiscus color 138 added to it—may be concentrated into hibiscus color concentrate 124 using atmospheric or vacuum evaporation or by osmosis, such as for example, reverse osmosis. The concentrate 124 comprises hibiscus color concentrated within a range of about 20% to about 60% by weight.

### Hibiscus Color Extraction Using Batch Processing

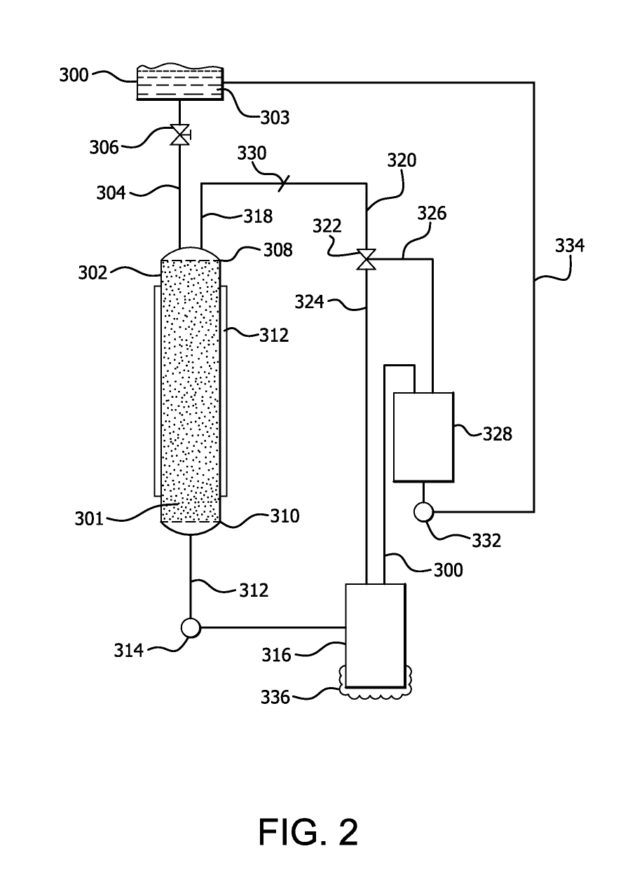

In an alternative embodiment shown in FIG. 2, batch processing, such as, for example, a batch percolating system, may be used in a process for extracting hibiscus color from hibiscus flowers. FIG. 2 illustrates a batch processing system which includes using solvent storage receptacle 300 in fluid communication with extraction column 302 along line 304. Line 304 includes valve 306 to control the flow of solvent 303 through line 304 and into column 302, or alternatively may be closed to stop the flow of solvent 303 down line 304. Column 302 is preferably loaded, between upper screen 308 and lower screen 310 with wet or dry, intact or ground hibiscus flowers 301. Steam heating jacket 312 may be used to cause solvent 303 which has been introduced into column 302 to percolate through wet or dry, intact or ground hibiscus flowers 301. Other heating means may, of course, be employed where desirable. Solvent 303 is used for extracting hibiscus color from wet or dry, intact or ground hibiscus flowers 301, and solvent having the extracted color dissolved therein will flow under the force of gravity or be pumped through lower screen 310 where it separates from the wet or dry, intact or ground hibiscus flowers 301 and travels into line 312 through which it is transported to pump 314 which, when activated, pumps the extracted hibiscus color to receptacle 316. Solvent vapor may escape from extraction column 302 through line 318 where it may condense. Once condensed, some of the solvent may fall back into extraction column 302 and some may enter line 320 where it will be directed by three-way valve 322 into either line 324 leading to product receptacle 316 or into line 326 leading to solvent recovery receptacle 328. If the pressure of solvent vapor in line 318 exceeds desirable levels, vapor may be released through pressure relief valve 330 to reduce the pressure. When it is desirable to produce a very dilute coloring composition, extra dilution may be accomplished by setting three-way valve 322 in such a manner as to direct additional solvent along line 324 into product receptacle 316 where it will dilute the dissolved coloring composition therein. When valve 322 directs the condensed solvent along line 326, the solvent accumulates in solvent recovery receptacle 328 from which it may be recirculated by pump 332 along line 334 to solvent storage receptacle 300.

When it is desirable to concentrate hibiscus color obtained from batch processing, solvent may be distilled from the hibiscus color by heating product receptacle 316, 336 to create a solvent vapor. This solvent vapor is removed from product receptacle 316 through line 300 in such a manner that the solvent condenses and accumulates in solvent recovery receptacle 328. Of course, the hibiscus color may be concentrated by merely allowing solvent to evaporate.

### Countercurrent Extraction of Hibiscus Color

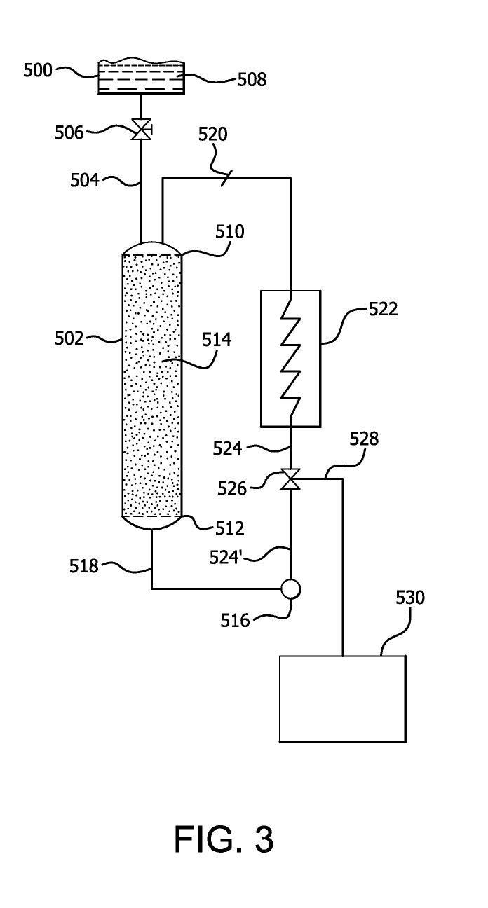

Referring now to FIG. 3, solvent extraction techniques, such as countercurrent extraction, may be used in a process for extracting hibiscus color from hibiscus flowers. For example, a countercurrent system, such as the one shown in FIG. 3, may be used for the extraction process. The system may comprise solvent storage receptacle 500 in fluid communication with extraction column 502 along line 504. Line 504 includes valve 506 for controlling solvent 508 flow through line 504 and into column 502, or alternatively may be closed to stop the flow of solvent 508 down line 504. Column 502 is preferably loaded, between upper screen 510 and lower screen 512 with wet or dry, intact or ground hibiscus flowers 514. Pump 516 drives solvent 508 along line 518 into column 502 and upward through wet or dry, intact or ground hibiscus flowers 514 where it extracts coloring compounds from wet or dry, intact or ground hibiscus flowers 514. Solvent 508 is continuously pushed upward through column 502 until it escapes through top screen 510 where it leaves behind wet or dry, intact or ground hibiscus flowers 514. Solvent 508 including extracted coloring compounds then enters line 520 which transports the solvent to heat exchanger 522. The solvent is constantly heated as it passes through heat exchanger 522 and into line 524 which conducts the solvent to three way valve 526 which may direct the solvent along line 528 to product container 530 or, alternatively, along line 524′ back to pump 516 which recirculates the solvent back through column 502 and, consequently, the rest of the system depicted in FIG. 3.

Preferred solvents for color extraction are food-grade organic solvents at a desirable polarity. The solvents are desirably tasteless or good-tasting. The solvents should have no adverse effects on coloring compounds. Solvents are also desirably volatile and inexpensive. Solvents with dielectric constants ranging from 1.97 to 78.00 at about 77° F. are preferred, especially solvents having dielectric constants between about 20.00 and 30.00 at about 77° F. Particular solvents useful in the practice of the invention include ethanol, liquid carbon dioxide, lower alcohols (about 2 carbons or more), ethers, ketones, and aldehydes, each being modified as necessary to achieve the desired polarity. In an alternative embodiment, lower alkyl and aryl solvents may be used, but must be fully removed from the hibiscus color if the compounds are intended for use in a consumable product. Ethanol, particularly in concentrations above 50%, and more particularly in a concentration of about 95%, is especially preferred. In producing food colorings, it is desirable to use a food-grade solvent which is generally recognized as safe (GRAS). Extraction time may range from about 10 minutes to about 3 days. A typical extraction time is about 4 hours to about 6 hours.

Extraction is likely at its greatest efficiency at a temperature at or near the boiling point of the solvent. In a non-limiting example, if pure or nearly pure ethanol is used as the solvent, the extraction temperature is about 172° F. to about 174° F. (at atmospheric pressure). However, the solvent does not need to be heated, and extraction can occur in a wide range of temperatures including ambient temperature. If desired, the solvent may be heated above its normal boiling point so long as sufficient pressure is applied to maintain the solvent in its liquid state.

The extracted color may be dilute or concentrated as desired. Additional solvent may be added to further dilute the extraction product if desired. Alternatively, solvent may be removed by conventional methods such as vacuum distillation to concentrate the extraction product.

### Stabilized Color Concentrate

Referring to FIGS. 1-3, hibiscus color 122, hibiscus color concentrate 124, residual hibiscus color 138, hibiscus color concentrate 140, any solution of hibiscus color (at any concentration) obtained from batch processing in receptacle 316 (as shown in FIG. 2), or countercurrent extraction in product container 530 (as shown in FIG. 3) may be stabilized with an agent into a color solution, whether or not that color solution is concentrated. In embodiments, the agent may be any sugar alcohol, including, but not limited to, a Polyol comprising from 2 to 12 carbons. For example, the agent may be any one of glycerol, propylene glycol, ethylene glycol, or the like. The pH value of the stabilized color concentrate 126, 142 or hibiscus solution obtained from batch processing or countercurrent extraction may be adjusted with food grade acids or bases in the range of about 2 to about 6. This stabilized concentrate color may be stored sealed in liquid form at a temperature ranging from about 34° F. to about 80° F. The stabilized color concentrate may be used as a color additive to food and drink products and as a coloring agent for drugs, cosmetics, and the like.

Referring to FIGS. 1-3, the solutions of hibiscus color of the invention including hibiscus color 122, residual hibiscus color 138, color concentrates 124, 140, and stabilized color concentrates 126, 142, hibiscus color solutions and concentrates obtained from batch processing in receptacle 316 (FIG. 2), or hibiscus color solutions obtained from countercurrent extraction in product container 530 (FIG. 3) may be dehydrated and dried into a hibiscus color powder or a paste, either of which may be anhydrous.

In an embodiment, compositions of hibiscus color powders or pastes may be produced from color solutions, concentrates, and stabilized concentrates by dry mixing, spray drying, freeze drying, blending, drying, extrusion or by any other convenient process. Additionally, compositions of hibiscus color powders or pastes may be prepared from color solutions, concentrates, and stabilized concentrates in a co-dried form with a bulking agent or the like. These compositions of powder or pastes and bulking agents may be produced by drying, mixing, co-spray drying, co-freeze drying, blending, co-drying, extrusion, or by any other convenient process. Bulking agents may be selected based on one or more functionalities which are desirable for use in product applications for which the hibiscus color powder or paste may be used. A broad range of bulking agents are compatible with hibiscus powders and pastes and can be selected. For example, bulking agents may be nutritive, such as, Maltodextrins or the like, or non-nutritive, such as, polydextrose or the like.

The spray drying may be completed through conventional mixing in which solutions of hibiscus color, hibiscus color concentrates, or stabilized color concentrates with bulking agents are formed. Alternatively, solutions of hibiscus color, hibiscus color concentrates, or stabilized color concentrates may be vacuum drum dried with one or more bulking agents. Any drying equipment conventionally used for the drying of color or dye solutions or other products having similar physical properties can be used to conduct such drying.

The amounts of hibiscus color 122, 142, hibiscus color concentrates 124, 140, or stabilized color concentrates 126, 142, whether or not commingled with bulking agents, or other ingredients, may be adjusted in order to reach the physical properties of the desired end product. For example, the desired bulk density of the resulting product may vary depending upon the end product (i.e., a coloring with bulking agent to be added to color juice drinks) or product form.

Compositions of hibiscus color powders or pastes whether or not commingled with bulking agents may be milled into a powder for use. Hibiscus color powder 129, 148 comprising hibiscus color with bulking agent, or hibiscus color paste may be stored under seal with or without desiccants.

While the invention is described in conjunction with specific embodiments, it is evident that many alternatives, modifications, permutations, and variations will become apparent to those skilled in the art in light of the foregoing description. Accordingly, it is intended that the invention embraces all such alternatives, modifications, permutations, and variations as falling within the scope of the claims below.

What is claimed is:

1. A water-based process for extracting hibiscus color from hibiscus flowers comprising:
soaking wet or dried hibiscus flowers in water;
digesting the soaking hibiscus flowers with a macerating enzyme to create a mixture of the hibiscus color and hibiscus solids; and
separating the hibiscus color from the hibiscus solids in the mixture.

2. The process of claim 1 wherein the soaking occurs with water at a temperature ranging from about 35° F. to about 210° F.

3. The process of claim 2 wherein the soaking occurs with water at a temperature of about 160° F.

4. The process of claim 2 wherein the soaking occurs for a time ranging from about one minute to about 20 minutes.

5. The process of claim 1 wherein the process comprises grinding wet or dried hibiscus flowers into particles in a size range of about 0.1 mm to about 2.0 mm.

6. The process of claim 1 wherein any one of centrifugation, filtration, countercurrent extraction, or pressing is used for separating the hibiscus color from the hibiscus solids.

7. The process of claim 6 wherein the hibiscus color comprises water, acid, color, and antioxidants.

8. The process of claim 7 wherein the acid comprises one or more of malic acid, citric acid, or oxalic acid.

9. The process of claim 6 wherein the process comprises soaking the solids in water to extract residual hibiscus color from the solids.

10. The process of claim 9 wherein any one of centrifugation, filtration, countercurrent extraction, or pressing is used for separating the residual hibiscus color from the solids.

11. The process of claim 10 wherein the process comprises combining the residual hibiscus color separated from the solids with the hibiscus color.

12. The process of claim 6 wherein the process comprises concentrating the hibiscus color into a concentrate.

13. The process of claim 12 wherein the process comprises drying the concentrate into a paste or powder of hibiscus color.

14. The process of claim 13 wherein the process comprises mixing the concentrate with a stabilizing agent to create a solution at a pH value in the range of about 2 to about 6.

15. The process of claim 14 wherein the stabilizing agent is a sugar alcohol comprising 2 to 12 carbons.

16. The process of claim 1 wherein the process comprises drying the hibiscus color into a paste or powder.

17. The process of claim 1 wherein the hibiscus color is used as a colorant for food products and non-food stuffs.

18. A process for extracting food-grade color from hibiscus flowers comprising:
digesting the hibiscus flowers soaked in water with macerating enzymes to create a color mixture;
separating a color solution from solids of the color mixture; and
concentrating the color solution into a color concentrate.

19. The process of claim 18 wherein the water is maintained at a temperature ranging from about 35° F. to about 210° F.

20. The process of claim 18 wherein the hibiscus flowers are soaked for a time period of about one minute to about 20 minutes.

21. The process of claim 18 wherein the color solution comprises water, acid, color, and antioxidants.

22. The process of claim 18 wherein the process comprises stabilizing the concentrate with a stabilizing agent.

23. The process of claim 22 wherein the stabilizing agent comprises a sugar alcohol having 2 to 12 carbons.

24. The process of claim 22 wherein the concentrate is stabilized at a pH value ranging from about 2 to about 6.

25. The process of claim 18 wherein the solids are mixed with water to extract residual color from the solids.

26. The process of claim 25 wherein the residual color is separated from the solids using any one of centrifugation, filtration, countercurrent extraction, or pressing.

27. The process of claim 26 wherein the residual color is combined with the color mixture.

28. The process of claim 18 wherein the process comprises drying the color solution into a powder or paste.

29. The process of claim 18 wherein the concentrate is used as a coloring for food, drinks, drugs, or cosmetics.

30. A process for extracting a stable color from hibiscus flowers comprising:
soaking the hibiscus flowers in water;
digesting the hibiscus flowers with a macerating enzyme to create a mixture; and
separating a stable color solution from solids in the mixture.

31. The process of claim 30 wherein the hibiscus flowers comprises dried flowers, wet flowers, or ground wet or dried flowers.

32. The process of claim 30 wherein the process comprises concentrating the stable color solution into a concentrate.

33. The process of claim 32 wherein the process comprises mixing the concentrate with a stabilizing agent to a pH value ranging from about 2 to about 6.

34. The process of claim 32 wherein the process comprises drying the concentrate into a powder or paste.

35. The process of claim 30 wherein the process comprises drying the stable color solution into a powder of paste.

36. A process for extracting color from hibiscus flowers comprising:
soaking hibiscus flowers in water;
extracting color from the hibiscus flowers; and
separating the color from hibiscus solids.

37. The process of claim 36 wherein the process comprises concentrating the color into a concentrate.

38. The process of claim 37 wherein the process comprises mixing the concentrate with a stabilizing agent to reach a pH value ranging from about 2 to about 6.

39. The process of claim 37 wherein the process comprises drying the concentrate into a powder or paste.

40. The process of claim 39 wherein a bulking agent is co-dried with the concentrate into a powder or paste.

41. The process of claim 36 wherein the process comprises drying the color into a powder or paste.

42. The process of claim 41 wherein a bulking agent is co-dried with the concentrate into a powder or paste.
